(19) United States
(12) Patent Application Publication
(10) Pub. No.: US 2012/0035553 A1
(43) Pub. Date: Feb. 9, 2012

(54) CLAMP FOR CLOSING FLEXIBLE TUBING BELONGING TO MEDICAL EQUIPMENT

(75) Inventors: Eugenio Lombardo, Ponte Nelle Alpi (IT); Aldo Calimeri, Capo d'Orlando (IT)

(73) Assignee: Gambro Lundia AB, Lund (SE)

(21) Appl. No.: 13/260,131

(22) PCT Filed: Feb. 10, 2010
(86) PCT No.: PCT/IB10/00254

§ 371 Date: Oct. 24, 2011

(30) Foreign Application Priority Data
Mar. 23, 2009 (IT) MI2009 A 000447
Mar. 23, 2009 (IT) MI2009 A 000450
Mar. 23, 2009 (IT) MI2009 A 000451

(51) Int. Cl. A61M 39/28

(52) U.S. Cl. 604/250

(57) ABSTRACT

A clamp for closing flexible tubing including a first arm having a first anchoring element, a second arm, a first connecting portion elastically connecting the first arm to the second arm, a second connecting portion connected to the second arm opposite the first connecting portion, extending towards the free end of the first arm, a second anchoring element positioned on the second connecting portion, a first projection integral to the first arm and facing the second arm, a second projection integral to the second arm and facing the first projection. The first and the second arms are movable between an open position, wherein the projections are in a spaced position, and a closed position, wherein the first anchoring element and the second anchoring element are coupled and the projections are in a close position squeezing a tubing.

The present invention relates to a clamp suitable for closing flexible tubing belonging to medical equipment.

In particular, the present invention relates to elastic clamps, in the specific field known by the English term “clamp”.

Clamps of this type are used in equipment for extra corporal blood treatment, such as dialysis and/or filtration equipment (haemodialysis, haemofiltration, haemodiafiltration, ultrafiltration), and serve for closing the flexible tubing belonging to such equipment by deforming it, in order to prevent the passage of fluids (for example blood, infusion or replacement fluids) contained in the tubing itself.

The closing of the tubing made by the above clamps is required, for example, in emergency situations or when moving to a subsequent step of the treatment in progress. For example, the flexible tubing is temporarily closed for changing an empty bag of fluid.

Clamps of known type are formed of a single plastic piece, which comprises a first arm, a second arm and a curved portion that elastically connects said two arms. A portion of the second arm opposite the curved portion extends towards a free end of the first arm and ends with a respective anchoring element. The free end of the first arm is provided with a respective anchoring element and with a manoeuvring portion. The two anchoring elements are set up for reciprocally engaging snap-wise when, acting with the fingers on the manoeuvring portion and on the second arm, the first arm is moved close to the second arm. The clamp further comprises two reciprocally facing projections and each one integral part of one of the two arms and has an opening obtained in the curved portion and an opening obtained in the portion of the second arm that extends towards the free end of the first arm. A flexible tubing is inserted in the two openings while the clamp is open, that is, while the two anchoring elements are not coupled, and it remains interposed between the two projections. By closing the clamp and coupling the anchoring elements, the projections reciprocally approach each other and squeeze the tubing up to fully closing the passage section of the same.

Clamps of the type described above are known for example from documents EP0995461 and EP0995462.

It is also known from document U.S. Pat. No. 3,822,052 a clamp for one-hand manipulation in controlling the flow of fluid through flexible tubing, such as intravenous tubing systems, and formed from a moulded flat strip of plastic material configured to provide hinge areas for bending the strip to provide a base with an overlying lever arm between which the tubing is disposed, and a slotted locking arm upstanding from the base and having notches to receive a locking head on the lever arm for cooperation with the notches to hold abutment surfaces on the lever arm and base in position for clamping the tubing there between in completely or partially constricted position, and with permissible finger manipulation causing relative movement between the lever and locking arms to effect quick release of the inter-engaged head and notches, respectively. Document WO 2004/1041343 discloses a clamp for closing flexible tubing belonging to medical equipment. It comprises a first arm having a first anchoring element positioned on a free end thereof and a second arm.

It also shows a first connecting portion connecting said first arm with said second arm and having a first opening for the passage of a flexible tubing; a second connecting portion connected to the second arm opposite the first connecting portion extends towards the free end of the first arm and has a second opening for the passage of the tubing.

A second anchoring element is positioned on the second connecting portion, a first projection is integral to the first arm and faces the second arm and a second projection is integral to the second arm and faces the first projection.

The flexible tubing passes through the first opening and the second opening remaining interposed between the first projection and the second projection, while the first arm and the second arm are movable between an open position, wherein the first anchoring element and the second anchoring element are released and the projections are in a spaced position, and a closed position wherein the first anchoring element and the second anchoring element are coupled and the projections are in a close position and squeeze the tubing.

The applicant has found that known clamps can be improved in many aspects from the functional and ergonomic point of view.

In particular, the Applicant has determined that known clamps offer poor usage practicality for the user.

In fact, in the first place during the closing of the clamp on the tubing, besides reciprocally approaching each other, the two arms tend to slide relative to one another laterally, that is, to misalign relative to a symmetry plane of the clamp, making the reciprocal engagement of the anchoring elements difficult.

Moreover the opening of the known clamp is sometimes difficult to manage.

Moreover, again during the closure, known clamps tend to slip in the user's hand, making the closing operation complicated and slowing it, whereas it should be carried out in the least possible time.

The Applicant has further noted that known clamps mark the tubing also when is they are open since the projections in any case press on the outside wall of the same. This is due to the fact that known clamps are made so that in their open and non-deformed configuration (without external constraints or forces applied to the clamp) the distance between the projections is in any case less than the tubing diameter, therefore when the tubing is inserted and spreads the arms, an elastic force closing the clamp is still present which releases on the piping and partially compresses it.

Within this scope, the technical task at the basis of the present invention is to provide a clamp suitable for closing flexible tubing belonging to medical equipment which should solve the drawbacks of the prior art mentioned above.

In particular, the object of the present invention is to provide a clamp capable of improving the functionality and making the use easier for the user.

A further object of the present invention is to propose a clamp which should cause the least possible damages to the flexible tubing whereon it is installed.

The above technical task and the specified objects are substantially achieved by a clamp suitable for closing flexible tubing belonging to medical equipment, comprising the technical features described in one or more of the annexed claims.

Further features and advantages of the present invention will appear more clearly from the exemplary and thus non limiting description of a preferred but non exclusive embodiment of a clamp suitable for closing hoses belonging to medical equipment, as illustrated in the annexed drawings, wherein:

FIG. 1 shows a front perspective view of a clamp according to the invention;

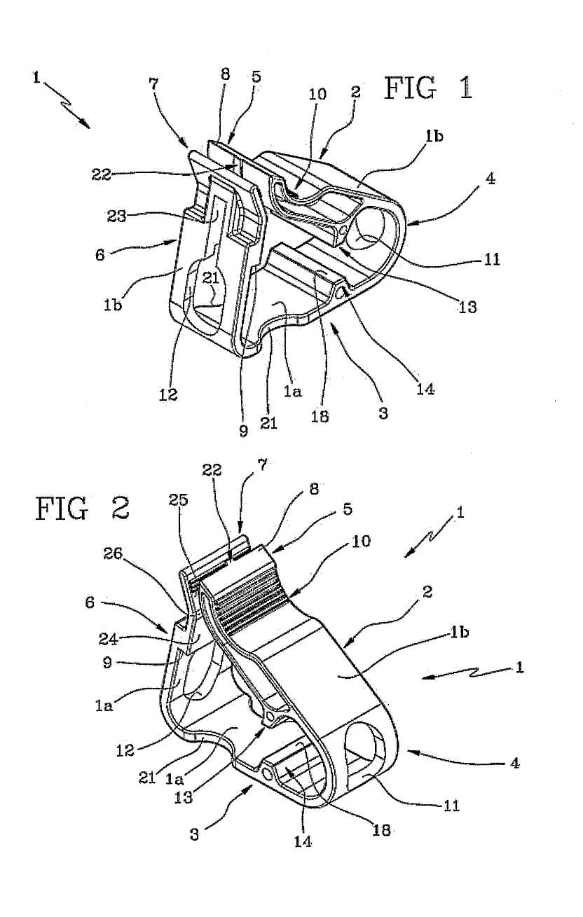

FIG. 2 shows a back perspective view of the clamp of FIG. 1;

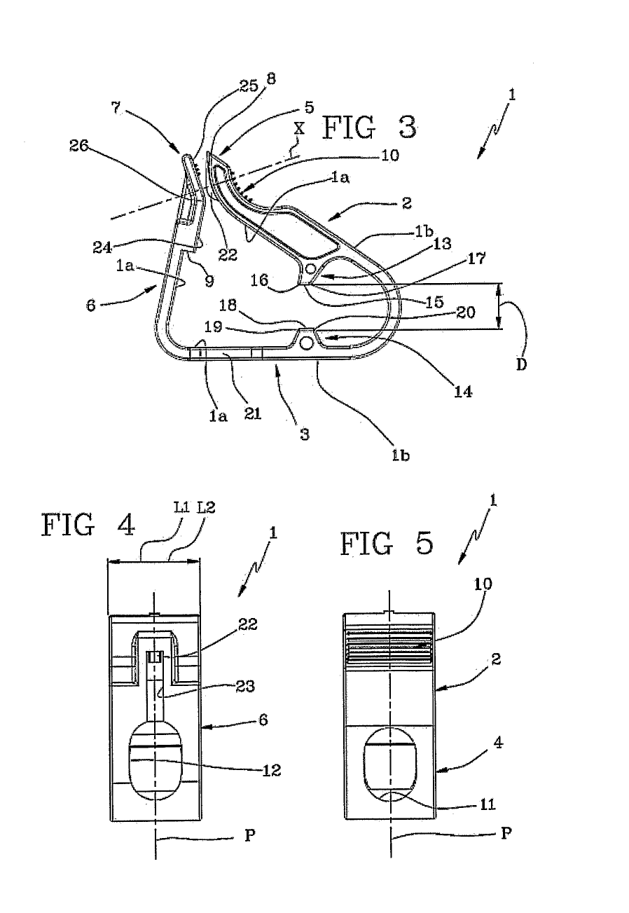

FIG. 3 shows a side elevation view of the clamp of FIGS. 1 and 2;

FIG. 4 shows a front elevation view of the clamp of FIGS. 1 and 2;

FIG. 5 shows a back elevation view of the clamp of FIGS. 1 and 2;

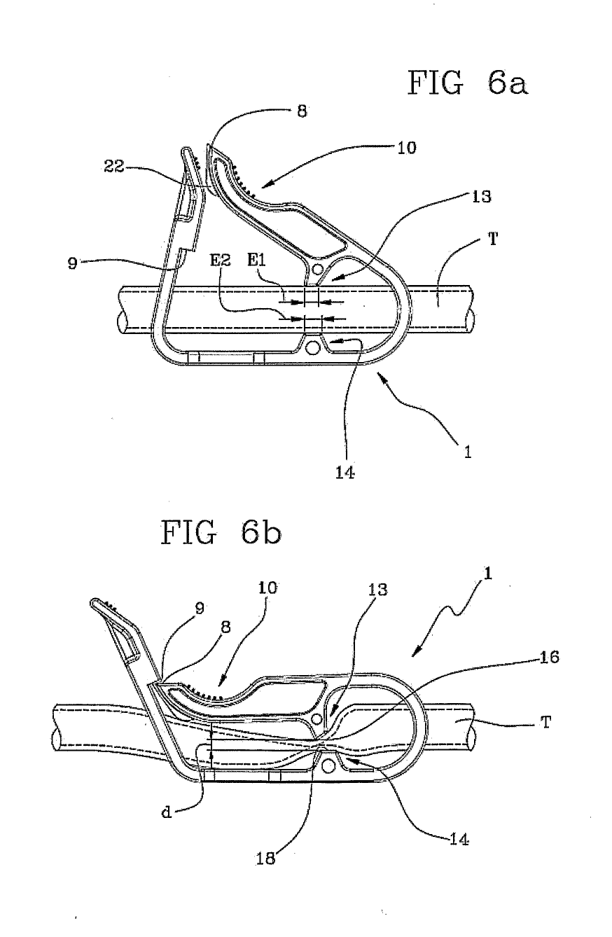

FIGS. 6a and 6b show the clamp of FIG. 2 associated to a flexile tubing in respective operating configurations.

In the annexed figures, reference numeral 1 indicates a clamp suitable for closing flexible tubing belonging to medical equipment according to the present invention.

Clamp 1 is optionally made in a single piece by moulding of plastic material, for example polypropylene, and is defined by a strip of material having a width larger than the thickness of the same.

The above strip comprises a first arm 2 integrally connected to a second arm 3 by means of a first connecting portion 4. In particular, the first arm 2 has a free end 5 and an opposite end integral to the first connecting portion 4. The second arm 3 has an end integral to the first connecting portion 4 and an opposite end integral to a second connecting portion 6 that extends towards the free end 5 of the first arm 2 and ends with a respective free end 7. The first arm 2 has a rectilinear development, the second arm 3 has a rectilinear development, the first connecting portion 4 has a circle arc development, the second connecting portion 6 has a rectilinear development. In a non deformed condition (FIGS. 1, 2 and 3), which as detailed hereinafter corresponds to an open position of clamp 1, the first arm 2 and the second arm 3 delimit an angle of about 45° with each other. Moreover, the second arm 3 and the second connecting portion 6 delimit an angle of about 80° with each other. In such non deformed condition, the free end 5 of the first arm 2 is further facing the free end 7 of the second connecting portion 6. In such condition, the strip is arranged ring-wise with its ends 5, 7 facing and has an internal peripheral surface 1a and an external peripheral surface 1b.

On the free end 5 of the first arm 2 there is a first anchoring element 8 which, in the embodiment shown, is an end portion of the same free end 5.

Moreover, on the second connecting portion 6 there is a second anchoring element 9 which, in the embodiment shown, is defined by a step positioned on the internal peripheral surface 1a of clamp 1 and is partially spaced from the free end 7 of the second portion 6. Step 9 delimits a stopping surface facing the second arm 3 and extends by the entire width of the second connecting portion 6.

The first arm 3 has a seat 10 obtained on the external peripheral surface 1b and close to the free end 5 of the first arm 2. Seat 10 is a recessing concavity relative to the remaining external peripheral surface 1b of the first arm 2 and has projections or knurls suitable for increasing the friction with the finger placed thereon.

By acting with the fingers of a hand, usually thumb and forefinger, on the second arm 3 and on seat 10 and moving the two arms 2, 3 close to one another, using the elastic deformation of the first portion 4 and of the second portion 6, the end portion 8 of the free end 5 of the first arm 2 is brought to step 9 and engaged snap-wise against the stopping surface (FIGS. 6a and 6b). In such condition, clamp 1 is closed.

The first connecting portion 4 has a first opening 11 and the second connecting portion 6 has a second opening 12, which openings 11, 12 substantially lie aligned along a direction parallel to the second arm 3.

The first arm 2 further carries a first projection 13 that develops from its own internal peripheral surface 1a towards the second arm 3. The second arm 3 carries a second projection 14 that develops from its own internal peripheral surface 1a towards the first arm 2. The first and the second projection 13, 14 are reciprocally facing.

The first projection 13 extends by the entire width of the first arm 2 and in a section parallel to a longitudinal centre line plane “P” of clamp 1, it has a shape tapered as a trapezium starting from the base towards the end. At the end, the first projection 13 optionally has a flat surface 15 delimited by two edges 16, 17. A first edge 16 facing the second connecting portion 6 and a second edge 17 facing the first connecting portion 4. The flat surface 15 of the first projection 13 has a width “L₁” comprised between about 10 mm and about 20 mm, optionally equal to about 14 mm. Moreover, the flat surface 15 of the first projection 13 has a longitudinal extension “E₁”, measured in a plane parallel to the longitudinal centre line plane “P” of clamp 1, comprised between about 1 mm and about 4 mm. The first edge 16 has a bending radius “r” comprised between about 0.2 mm and about 0.5 mm, optionally equal to about 0.3 mm.

The second projection 14 extends by the entire width of the second arm 3 and in a section parallel to a longitudinal centre line plane “P” of clamp 1, it has a shape tapered as a trapezium starting from the base towards the end. At the end, the second projection 14 has a flat surface 18 delimited by two edges 19, 20. The flat surface 18 of the second projection 14 has a width “L2” comprised between about 10 mm and about 20 mm, optionally equal to about 14 mm. Moreover, the flat surface 18 of the second projection 14 has a longitudinal extension “E₂”, measured in a plane parallel to the longitudinal centre line plane “P” of clamp 1, comprised between about 1 mm and about 4 mm.

While clamp 1 is open, with the anchoring elements 8, 9 released and projections 13, 14 spaced from each other, a flexible tubing “T” is inserted through the first and the second opening 11, 12 and thus a portion of the same tubing “T” remains inside clamp 1 and interposed between the two projections 13, 14 (FIG. 6a).

In such open position, the flat surface 15 of the first projection 13 and the flat surface 18 of the second projection 14 are parallel and abutted against opposite portions of the flexible tubing “T”.

Closing clamp 1, according to the description above, projections 13, 14 are moved close to each other and also rotated relative to each other. Such approach causes the squeezing of tubing “T”, up to closing the inside passage section of tubing “T” itself (FIG. 6b).

In the closed position of clamp 1, when the first and the second anchoring element 8, 9 are reciprocally coupled, the flat surface 18 of the second projection 14 still contacts tubing “T” whereas the first projection 13 presses on tubing “T” with its first edge 16, which therefore faces said flat surface 18.

In the open position, the first projection 13 and the second projection 14 are spaced by a distance “D” comprised between about 6 mm and about 7 mm, optionally equal to about 6.6 mm.

In the closed position, the first projection 13 and the second projection 14 are spaced by a minimum distance “d” comprised between about 0.2 mm and about 0.7 mm, optionally equal to about 0.5 mm.

Optionally, the second arm 3 has, at one or both its side edges, a recess 21 arranged in the proximity of the second connecting portion 6. Such recesses 21, which in the embodiment illustrated are symmetrical relative to the longitudinal centre line plane “P”, seat the user's forefinger while he/she manoeuvres clamp 1.

Clamp 1 comprises also a first guiding element 22 associated to the first arm 2 and a second guiding element 23 associated to the second arm 3.

The guiding elements 22, 23 engage reciprocally during the movement between the open position and the closed position of clamp 1 and prevent the side movement of the first arm 2 and of the second arm 3 relative to each other and relative to the longitudinal centre line plane of clamp 1 during the movement between the open and the closed positions.

It is to be noted that in the closed position of the first arm 2 and the second arm 3, the first guiding element 22 and the second guiding element 23 are disengaged (see FIG. 1). The above means that, in the closed position of the first arm 2 and the second arm 3, the first guiding element 22 and the second guiding element 23 allow the side movement of the first arm 2 and of the second arm 3 relative to a longitudinal centre line plane P of the clamp 1. This lateral slide movement can help in easy and quick disengagement of the two arms.

Optionally also in the open position of the first arm 2 and the second arm 3, the first guiding element 22 and the second guiding element 23 are disengaged. In other words in the open position the first guiding element 22 and the second guiding element 23 allow the side movement of the first arm 2 and of the second arm 3 relative to a longitudinal centre line plane P of the clamp 1.

In the illustrated embodiment, the first guiding element 22 is a tab positioned at the free end 5 of the first arm 2 on the internal surface 1a and in a zone opposite seat 10 for the thumb. Tab 22 extends parallel to the longitudinal centre line plane “P” and has an arched edge.

The second guiding element 23 is a groove obtained in the second connecting portion 6 at the free end 7. More in detail, groove 23 develops along the longitudinal centre line plane “P”, optionally as continuation of the second opening 12. Groove 23 has such a width as to seat tab 22 but prevent or at least limit side movements of the latter, or movements orthogonal to the longitudinal centre line plane “P” of clamp 1 at least during the engaging movement.

According to a not shown embodiment, the elements are inverted, that is, the tab is positioned at the free end 7 of the second connecting portion 6 and groove 23 is obtained at the free end 5 of the first arm 2.

Optionally, groove 23 is interposed between the free end 7 of the second connecting portion 6 and step 9. In fact, step 9 is positioned in the proximity of an end of groove 23 close to the second opening 12. In the embodiment shown, step 9 is positioned at the zone of junction of groove 23 into the second opening 12.

Step 9 separates the internal peripheral surface 1a belonging to the second connecting portion 6 into two surfaces lying on staggered planes. The internal surface 1a interposed between step 9 and the free end 7 is in turn divided into a first surface 24 parallel to the remaining internal surface 1a but closer to the first arm 2 and a second internal surface 25 inclined relative to the first surface 24. The second inclined surface 25 develops towards the external peripheral surface 1b of the same second connecting portion 6, that is, away from the free end 5 of the first arm 2, and with the first surface 24 it forms an edge 26 and an angle of about 160°.

Groove 23 develops in the second connecting portion 6 at the first surface 24 and partially, in the second internal inclined surface 25, without reaching the free end 7.

Moreover, the positioning zone of tab 22 located on the first arm 2 has a concave and arched shape that in the open position of clamp 1, faces the inclined surface 25 mentioned above.

In use, in the open position of clamp 1, visible in FIGS. 1-5, at least a part of the support seat 10, tab 22 and groove 23 are reciprocally aligned along an initial movement direction “X” of arms 2 and 3.

In such position, while the flexible tubing “T” is inserted in clamp 1 and is open, since projections 13, 14 are spaced from each other, tab 22 faces the inclined surface 25 and external to groove 23 (FIG. 6a). Projections 13, 14 slightly compress tubing “T” so as to hold clamp 1 on tubing “T” itself.

When the user pushes the free end 5 of the first arm 2 towards the second connecting portion 6, the concave and arched positioning zone of tab 22 rests against the inclined surface 25 and tab itself 22 enters into groove 23. The concave and arched zone slides on the inclined surface 25 and then on the first surface 24 and tab 22 slides into groove 23 until the free end 5 of the first arm 2 reaches step 9, where it engages snap-wise against the stopping surface (FIG. 6b, closed position of clamp 1), using the elasticity of the second connecting portion 6. Tab 22 in the meantime has come out of groove 23.

To open clamp 1, it is sufficient to manually move the free end 7 of the second connecting portion 6 away from the first arm 2, optionally provided with rises or knurls. The first arm 2, thanks to the elastic return of the first connecting portion 4 and to the elastic return of the flexible tubing “T” interposed between projections 13, 14, returns to the open position.

Thanks to the guiding elements (tab 22 and groove 23), the reciprocal side movement of the closing arms is prevented, or in any case strongly limited. As a consequence, the closing movement of the clamp is more accurate and easy to be carried out by the user.

The presence of the guiding elements further allows making the clamp so that, when the clamp is open in the rest position, that is, is not subject to any constraint or external force (the flexible tubing is not present which in known clamps, keeps the projections spread), the distance between the projections is much larger than that of the prior art clamps and substantially equal, a little smaller, than the outside diameter of tubing “T”. In this situation when the tubing is inserted, the elastic closing of the clamp open on the tubing itself exerts a much lighter force than that exerted by the known clamps and marks the tubing less.

The movement of the first arm 2 relative to the second arm 3 for achieving the lock certainly is greater than in known type clamps but such movement is advantageously guided by the guiding elements.

Also the shape of projections 13 and 14 contributes to decreasing the impression of the same on the tubing when the clamp is open, since the flat surfaces 15, 18 allow increasing the contact area with the tubing itself.

Seat 10 for the thumb together with recesses 21 for the forefinger ensure a safer grip for the user and thus increase the usage practicality.

1.-29. (canceled)

30. A clamp for closing flexible tubing belonging to medical equipment, comprising:
a first arm having a first anchoring element positioned on a free end thereof;
a second arm;
a first connecting portion elastically connecting said first arm with said second arm and having a first opening for the passage of a flexible tubing;
a second connecting portion connected to the second arm opposite the first connecting portion, extending towards the free end of the first arm and having a second opening for the passage of said flexible tubing;
a second anchoring element positioned on the second connecting portion;
a first projection integral to the first arm and facing the second arm;
a second projection integral to the second arm and facing the first projection; the flexible tubing passing through the first opening and the second opening remaining interposed between the first projection and the second projection;
the first arm and the second arm being movable between an open position, wherein the first anchoring element and the second anchoring element are released and the projections are in a spaced position, and a closed position, wherein the first anchoring element and the second anchoring element are coupled and the projections are in a close position and squeeze the tubing for completely closing a passage section of said tubing;
a first guiding element associated to the first arm and
a second guiding element associated to the second arm; said guiding elements reciprocally engaging during the movement between the open position and the closed position and preventing the side movement of the first arm and of the second arm relative to a longitudinal centre line plane of the clamp, wherein in the closed position of the first arm and the second arm the first guiding element and the second guiding element are disengaged.

31. The clamp according to claim 30, wherein the first guiding element is a tab positioned at the free end of the first arm and the second guiding element is a groove obtained at a free end of the second connecting portion.

32. The clamp according to claim 30, wherein the first guiding element is a groove obtained at the free end of the first arm and the second guiding element is a tab positioned at a free end of the second connecting portion.

33. The clamp according to claim 30, wherein the first arm has a support seat for a finger of the user obtained at the free end of said first arm and wherein the first guiding element is positioned on a surface of the first arm close to the free end and opposite said support seat.

34. The clamp according to claim 30, wherein the support seat is a recessing concavity relative to a remaining external surface of the first arm.

35. The clamp according to claim 34, wherein at least in the open position of the clamp, at least a part of the support seat, the first guiding element and the second guiding element are reciprocally aligned along a direction of initial movement of said arms.

36. The clamp according to claim 30, wherein the second guiding element is interposed between the free end of the second connecting portion and the second anchoring element.

37. The clamp according to claim 31, wherein the groove is an extension of the second opening.

38. The clamp according to claim 30, wherein the first anchoring element is an end portion of said free end and the second anchoring element is a step.

39. The clamp according to claim 38, wherein the step is positioned in the proximity of an end of the groove close to the second opening.

40. The clamp according to claim 30, wherein in the closed position of the first arm and the second arm, the first guiding element and the second guiding element allow the side movement of the first arm and of the second arm relative to a longitudinal centre line plane of the clamp.

41. The clamp according to claim 30, wherein in the open position of the first arm and the second arm, the first guiding element and the second guiding element the first guiding element and the second guiding element are disengaged.

42. The clamp according to claim 30, wherein in the open position of the first arm and the second arm, the first guiding element and the second guiding element allow the side movement of the first arm and of the second arm relative to a longitudinal centre line plane of the clamp.

43. The clamp according to claim 30, wherein the second arm has at least one recess shaped on a side edge thereof, for seating the user's forefinger.

44. The clamp according to claim 43, wherein the second arm has two recesses, each shaped on a respective side edge.

45. The clamp according to claim 44, wherein the two recesses are symmetrical relative to a longitudinal centre line plane of the clamp.

46. The clamp according to claim 43, wherein the recess or the two recesses is/are arranged in the proximity of the second connecting portion.

47. The clamp according to claim 30, wherein the first arm has a support seat for the user's thumb obtained at the free end of said first arm.

48. The clamp according to claim 30, wherein the support seat is a recessing concavity relative to a remaining external surface of the first arm.

49. The clamp according to claim 30, wherein the first projection and the second projection have each a flat surface, said flat surfaces, when the first arm and the second arm are in the open position, being reciprocally parallel and abutted against the flexible tubing.

50. The clamp according to claim 49, wherein the first projection has an edge and wherein said edge, when the first arm and the second arm are in the closed position, faces the flat surface of the second projection and acts against the flexible tubing.

51. The clamp according to claim 49, wherein the flat surface of the first projection has a longitudinal extension comprised between about 1 mm and about 3 mm.

52. The clamp according to claim 49, wherein the flat surface of the second projection has a longitudinal extension comprised between about 1 mm and about 4 mm.

53. The clamp according to claim 49, wherein the flat surface of the first projection has a width comprised between about 10 mm and about 20 mm.

54. The clamp according to claim 49, wherein the flat surface of the second projection has a width comprised between about 10 mm and about 20 mm.

55. The clamp according to claim 50, wherein the edge of the first projection has a bending radius comprised between about 0.2 mm and about 0.5 mm.

56. The clamp according to claim 30, wherein when the first arm and the second arm are in their closed position, the minimum distance between the two projections is comprised between about 0.2 mm and about 0.7 mm.

57. A clamp for closing flexible tubing belonging to medical equipment, comprising:
a first arm having a first anchoring element positioned on a free end thereof;
a second arm;
a first connecting portion elastically connecting said first arm with said second arm and having a first opening for the passage of a flexible tubing;
a second connecting portion connected to the second arm opposite the first connecting portion, extending towards the free end of the first arm and having a second opening for the passage of said flexible tubing;
a second anchoring element positioned on the second connecting portion;
a first projection integral to the first arm and facing the second arm;
a second projection integral to the second arm and facing the first projection; the flexible tubing passing through the first opening and the second opening remaining interposed between the first projection and the second projection;
the first arm and the second arm being movable between an open position, wherein the first anchoring element and the second anchoring element are released and the projections are in a spaced position, and a closed position, wherein the first anchoring element and the second anchoring element are coupled and the projections are in a close position and squeeze the tubing for completely closing a passage section of said tubing, wherein the second arm has at least one recess shaped on a side edge thereof, for seating the user's forefinger, characterised in that the recess is arranged in the proximity of the second connecting portion.

58. The clamp according to claim 57, wherein the second arm has two recesses, each shaped on a respective side edge.

59. The clamp according to claim 58, wherein the two recesses are symmetrical relative to a longitudinal centre line plane of the clamp.

60. The clamp according to claim 57, wherein the recess or the two recesses is/are arranged in the proximity of the second connecting portion.

61. The clamp according to claim 57, wherein the first arm has a support seat for the user's thumb obtained at the free end of said first arm.

62. The clamp according to claim 61, wherein the support seat is a recessing concavity relative to a remaining external surface of the first arm.

63. A clamp for closing flexible tubing belonging to medical equipment, comprising:
a first arm having a first anchoring element positioned on a free end thereof;
a second arm;
a first connecting portion elastically connecting said first arm with said second arm and having a first opening for the passage of a flexible tubing;
a second connecting portion connected to the second arm opposite the first connecting portion, extending towards the free end of the first arm and having a second opening for the passage of said flexible tubing;
a second anchoring element positioned on the second connecting portion;
a first projection integral to the first arm and facing the second arm;
a second projection integral to the second arm and facing the first projection; the flexible tubing passing through the first opening and the second opening remaining interposed between the first projection and the second projection;
the first arm and the second arm being movable between an open position, wherein the first anchoring element and the second anchoring element are released and the projections are in a spaced position, and a closed position, wherein the first anchoring element and the second anchoring element are coupled and the projections are in a close position and squeeze the tubing for completely closing a passage section of said tubing; wherein the first projection and the second projection have each a flat surface, said flat surfaces, when the first arm and the second arm are in the open position, being reciprocally parallel and abutted against the flexible tubing.

64. The clamp according to claim 63, wherein the first projection has an edge and wherein said edge, when the first arm and the second arm are in the closed position, faces the flat surface of the second projection and acts against the flexible tubing.

65. The clamp according to claim 63, wherein the flat surface of the first projection has a longitudinal extension comprised between about 1 mm and about 3 mm.

66. The clamp according to claim 63, wherein the flat surface of the second projection has a longitudinal extension comprised between about 1 mm and about 4 mm.

67. The clamp according to claim 63, wherein the flat surface of the first projection has a width comprised between about 10 mm and about 20 mm.

68. The clamp according to claim 63, wherein the flat surface of the second projection has a width comprised between about 10 mm and about 20 mm.

69. The clamp according to claim 64, wherein the edge of the first projection has a bending radius comprised between about 0.2 mm and about 0.5 mm.

70. The clamp according to claim 63, wherein when the first arm and the second arm are in their closed position, the minimum distance between the two projections is comprised between about 0.2 mm and about 0.7 mm.
